(19) United States
(12) Patent Application Publication
(10) Pub. No.: US 2022/0169203 A1
(43) Pub. Date: Jun. 2, 2022

(54) VEHICLE MONITORING SYSTEM

(71) Applicant: EGK TECHNOLOGY CO., LTD., Kaohsiung City (TW)

(72) Inventor: Jia-Wei CHEN, Kaohsiung City (TW)

(21) Appl. No.: 17/109,644

(22) Filed: Dec. 2, 2020

(51) Int. Cl. B60R 25/25; G08G 1/00; H04N 5/247; G06K 9/00; H04N 7/18; A61B 5/0205; A61B 5/1455; A61B 5/00; A61B 5/1171; G07C 9/37

(52) U.S. Cl. CPC B60R 25/25; A61B 5/021; H04N 5/247; G06K 9/00832; G06K 9/00288; G06K 9/00268; G06K 9/00885; G06K 9/00791; H04N 7/181; A61B 5/02055; A61B 5/14551; A61B 5/4845; A61B 5/1176; G07C 9/37; G06K 2009/00932; G08G 1/202

(57) ABSTRACT

A vehicle monitoring system adapted to manage and monitor vehicles includes a management platform and a control device connected thereto and installed in each vehicle. The control device includes a processor where predetermined conditions set as standards of safe driving are stored, a camera lens set adapted to capture actual images, a storage unit adapted to store the captured images, and a reminding unit connected to the processor. The processor compares the captured images with the predetermined conditions, and a comparison result of each vehicle is sent to the management platform for recording and monitoring. If the captured images do not fulfill the predetermined conditions, an abnormal state is shown, and the reminding unit reminds the vehicle driver to reduce the incidence of traffic accidents. The management platform monitors the driving state of vehicles proactively according to the comparison result to enhance occupational safety and emergency response capability.

### BACKGROUND OF THIS INVENTION

### 1. Field of this invention

This invention relates to a monitoring system and relates particularly to a vehicle monitoring system.

### 2. Description of the Related Art

Generally, causes of traffic accidents are closely related to a driving state of a vehicle driver. For instance, poor driving states maybe caused by poor physiological states of the vehicle driver which may include low blood pressure, low blood sugar, drunk-driving, fatigue-driving and so on, and that may greatly affect the concentration of the vehicle driver during a driving process and likely to cause a traffic accident. Professional vehicle drivers usually drive large vehicles such as buses or trucks except taxi drivers. Large vehicles will cause more serious damage and casualty when a traffic accident happens. Therefore, if the physiological state of the vehicle driver can be detected and monitored properly before the vehicle driver drives the vehicle, unnecessary traffic accidents can be reduced. Freight forwarders usually monitor the physiological state of each vehicle driver by measuring and recording the physiological state of each vehicle driver compulsorily via relevant devices before the vehicle driver picks a vehicle. However, owing to lack of manpower, the vehicle driver usually measures the physiological state by himself, and that is difficult to manage the actual physiological state of the vehicle driver since the vehicle driver may fill in the incorrect physiological data. If a person devoted to measure and record the physiological state of each vehicle drivers is hired, it will increase labor costs, and that requires to be improved.

After the vehicle driver picks the vehicle, GPS positioning system is usually adapted to track the driving path and staying time of the vehicle. However, the physiological state of the vehicle driver and the instant driving state of the vehicle during the driving process are unable to be known. For example, it is unable to know if the vehicle driver keeps a safety distance from another vehicles, or if the vehicle driver deviates from the lane. The driving behavior of the vehicle driver can be known passively only from complaints of other vehicle drivers or passersby, violation tickets, or notifications after a traffic accident happens. Thus, the driving state of the vehicle driver cannot be monitored immediately and the driving behavior of the vehicle driver cannot be managed proactively. That needs to be improved.

### SUMMARY OF THIS INVENTION

The object of this invention is to provide a vehicle monitoring system capable of monitoring and managing a driving state of each vehicle proactively to enhance occupational safety and emergency response capability.

The vehicle monitoring system of this invention includes a management platform adapted to monitor and manage a plurality of vehicles and a control device connected to the management platform and installed in each vehicle. The control device has a processor disposed in each vehicle and being in communication with the management platform, a camera lens set disposed in each vehicle and connected to the processor, a storage unit connected to the processor and adapted to store a plurality of actual images captured by the camera lens set, and a reminding unit connected to the processor. The processor in which a plurality of predetermined conditions set as standards of safe driving are stored is configured to compare the actual images of the camera lens set with the predetermined conditions to obtain a comparison result serving to check a driving state of the vehicle. The comparison result is delivered to the management platform by the processor and recorded in the management platform. If the actual images does not fulfill the predetermined conditions, an abnormal state of the comparison result is shown, and the reminding unit reminds the vehicle driver to thereby reduce an incidence of traffic accidents. The management platform monitors the driving state of vehicles proactively according to the comparison result and allows administrator to contact with the vehicle driver when an abnormal state of the comparison result is shown to thereby enhance occupational safety and emergency response capability. Further, a vehicle dispatching device is in communication with the management platform to automatically detect an identity and a physiological state of the vehicle driver before the vehicle driver drive the vehicle to thereby increase the driving safety and reduce labor costs.

Preferably, an electronic control system is installed in each vehicle and a signal line is connected to the processor and the electronic control system for accessing vehicle driving data of the electronic control system and delivering the vehicle driving data to the processor.

Preferably, the camera lens set includes a front camera, a left camera, a right camera, a rear camera, and an interior camera connected to the processor respectively for reflecting different sides of each vehicle. The actual images include a first image photographically captured by the front camera for reflecting a front side of the vehicle, a second image photographically captured by the left camera for reflecting a left side of the vehicle, a third image photographically captured by the right camera for reflecting a right side of the vehicle, a fourth image photographically captured by the rear camera for reflecting a rear side of the vehicle, and a fifth image photographically captured by the interior camera for reflecting a vehicle driver inside the vehicle.

Preferably, a vehicle dispatching device is in communication with the management platform. The vehicle dispatching device includes a controller in communication with the management platform, a feature recognition set connected to the controller and adapted to detect feature data of a vehicle driver, a physiological state recognition set connected to the controller and adapted to detect physiological data of the vehicle driver, and a key cabinet controlled by the controller and configured to accommodate at least one key. The controller includes a feature management module in which a plurality of feature default values are stored and a physiological management module in which a plurality of physiological default values are stored. The feature management module is connected to the feature recognition set and adapted to receive the feature data of the feature recognition set and compare the feature data with the feature default values to obtain a feature result serving to check an identity of the vehicle driver. The physiological management module is connected to the physiological state recognition set and adapted to receive the physiological data of the physiological state recognition set and compare the physiological data with the physiological default values to obtain a physiological result serving to check a physiological state of the vehicle driver. An opening operation and a close operation of the key cabinet are controlled by the controller according to the feature result and then the physiological result. The feature result and the physiological result are sent from the controller to the management platform and recorded in the management platform.

Preferably, the feature recognition set includes a facial features recognition device.

Preferably, the feature recognition set includes a digital vein recognition device.

Preferably, the physiological state recognition set includes a blood measuring device adapted to measure blood pressure and blood oxygen of the vehicle driver.

Preferably, the physiological state recognition set includes a sobriety testing device.

Preferably, the physiological state examination set includes a body temperature measuring device.

Preferably, the controller includes a vehicle management module in communication with the management platform and adapted to display a dispatching task to the vehicle driver.

### BRIEF DESCRIPTION OF THE DRAWINGS

FIG. 1 is a schematic view showing a first preferred embodiment of this invention;

FIG. 2 is a schematic view showing the management platform is in communication with the control device installed in each vehicle;

FIG. 3 is a schematic view showing an operation of the first preferred embodiment of this invention;

FIG. 4 is a schematic view showing a second preferred embodiment of this invention; and

FIG. 5 is a schematic view showing an operation of the second preferred embodiment of this invention.

### DETAILED DESCRIPTION OF THE PREFERRED EMBODIMENTS

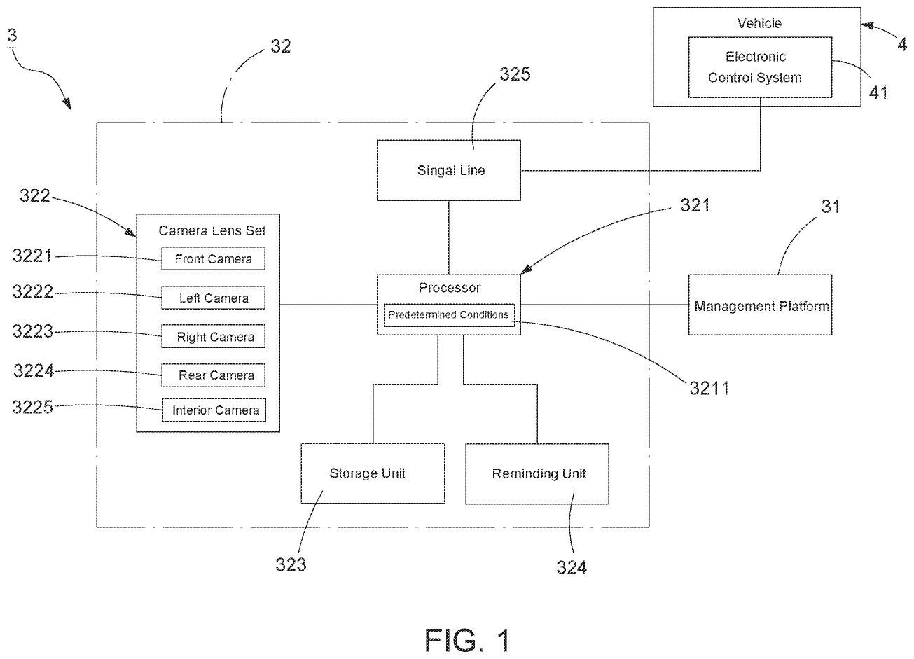

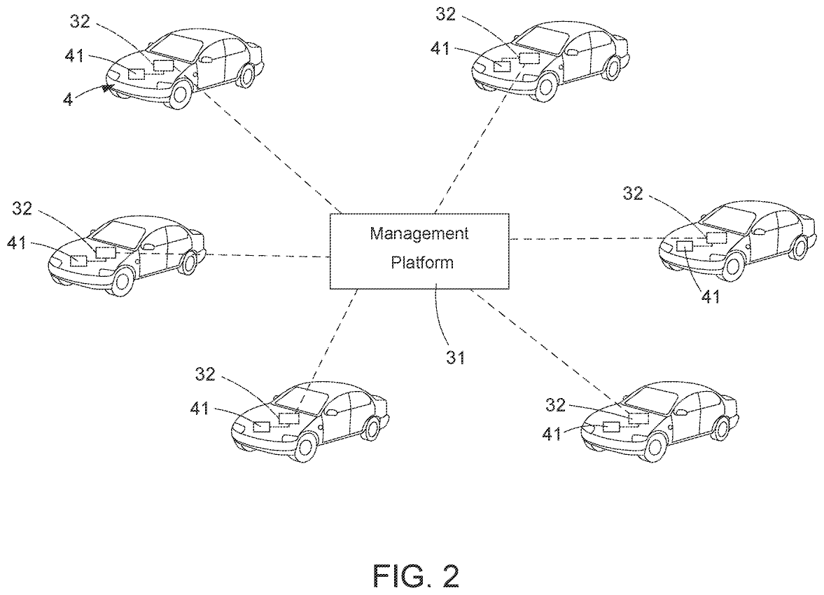

Referring to FIG. 1 and FIG. 2, a vehicle monitoring system 3 of a first preferred embodiment of this invention comprises a management platform 31 adapted to monitor and manage a plurality of vehicles 4 and a control device 32 connected to the management platform 31 and installed in each vehicle 4.

The control device 32 includes a processor 321 disposed in each vehicle 4 and being in communication with the management platform 31, a camera lens set 322 disposed in each vehicle 4 and connected to the processor 321, a storage unit 323 connected to the processor 321 and adapted to store a plurality of actual images captured by the camera lens set 322, and a reminding unit 324 connected to the processor 321. In this preferred embodiment, the camera lens set 322 has a front camera 3221, a left camera 3222, a right camera 3223, a rear camera 3224, and an interior camera 3225 connected to the processor 321 respectively for reflecting different sides of each vehicle 4. The actual images includes a first image photographically captured by the front camera 3221 for reflecting a front side of the vehicle 4, a second image photographically captured by the left camera 3222 for reflecting a left side of the vehicle 4, a third image photographically captured by the right camera 3223 for reflecting a right side of the vehicle 4, a fourth image photographically captured by the rear camera 3224 for reflecting a rear side of the vehicle 4, and a fifth image photographically captured by the interior camera 3225 for reflecting a vehicle driver inside the vehicle 4. Further, the reminding unit 324 can send out an alarm with sound or light to thereby remind the vehicle driver. A plurality of predetermined conditions 3211 are stored in the processor 321. The predetermined conditions 3211 are set as standards for safe driving. The processor 321 is configured to compare the actual images captured by the camera lens set 322 with the predetermined conditions 3211 to obtain a comparison result serving to check a driving state of the vehicle. The comparison result is delivered to the management platform 31 by the processor 321 and recorded in the management platform 31. The management platform 31 monitors the driving state of each vehicle 4 proactively according to the comparison result. For instance, it can be known whether a safety distance is kept between the driving vehicle 4 with another vehicles, whether the driving vehicle 4 exceeds the speed limit, or whether the driving vehicle 4 is deviated from the lane according to the comparison result. If the actual images do not fulfill the predetermined conditions 3211, an abnormal state of the comparison result is shown, and the reminding unit 324 reminds the vehicle driver to reduce the incidence of traffic accidents. In this preferred embodiment, an electronic control system 41 is installed in each vehicle 4 and a signal line 325 is connected to the processor 321 and the electronic control system. 41 for accessing vehicle driving data of the electronic control system 41 and delivering the vehicle driving data to the processor 321. The vehicle driving data may include the actual driving speed of the vehicle 4, proofs of using left turn signal or right turn signal that can assist in the determination of the processor 321.

For instance, when the front camera 3221 captures a first image which reflects the front side of the vehicle 4 and displays the lane departure of the vehicle 4, the first image is sent to the processor 321 and compared with the predetermined conditions 3211 to obtain a comparison result. When the processor 321 determines that the vehicle 4 is deviated from the lane, namely the actual images do not fulfill the predetermined conditions 3211, the processor 321 then reads the vehicle driving data of the electronic control system 41 via the signal line 325 to determine if a turn signal is used. If the turn signal is used, the processor 321 determines that a normal state of the comparison result is shown. Alternatively, if the turn signal is not used, the processor 321 determines that an abnormal state of the comparison result is shown. When an abnormal state of the comparison result is shown, the processor 321 will actuate the reminding unit 324 to remind the vehicle driver. Further, all comparison results will be delivered and recorded in the management platform 31 to allow proactive monitoring.

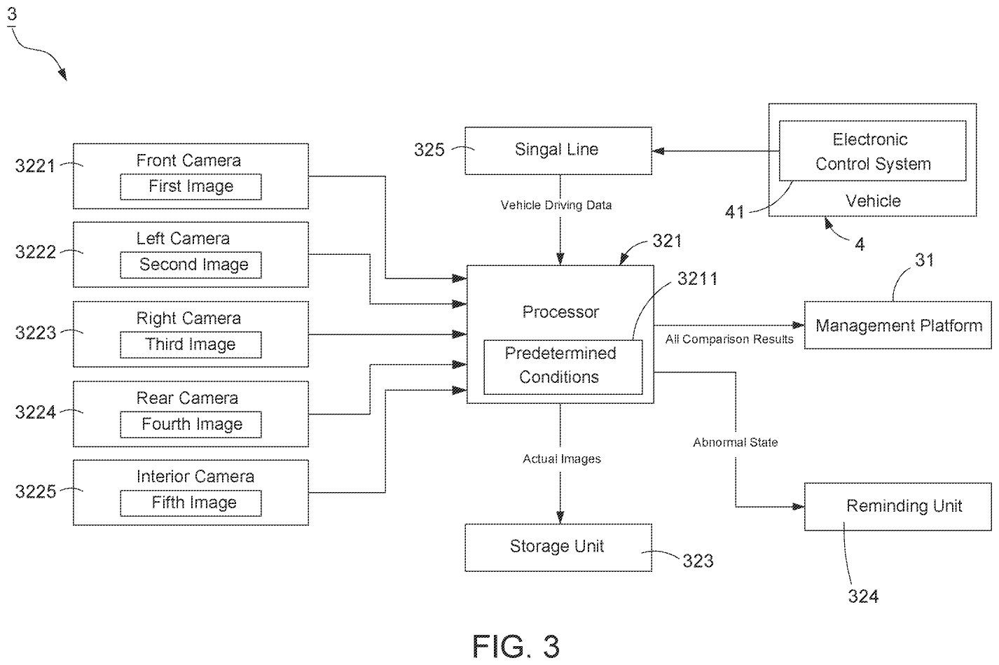

Referring to FIG. 1, FIG. 2 and FIG. 3, when the vehicle monitoring system 3 is adapted to monitor each vehicle 4, the actual images captured by the camera lens set 322 are delivered to the processor 321 which is installed in each vehicle 4. The processor 321 then compares the actual images with the predetermined conditions 3211 to obtain the comparison result. Simultaneously, the actual images are delivered and stored in the storage unit 323. The processor 321 then reads the vehicle driving data of the electronic control system 41 via the signal line 325 so as to determine if an abnormal state of the comparison result is shown. When an abnormal state of the comparison result is shown, the reminding unit 324 then sends out an alarm to remind the vehicle driver to thereby increase the attention of the vehicle driver. Further, all comparison results including normal states and abnormal states of the comparison results will be transmitted and recorded in the management platform 31. Proactive monitoring of the driving state of each vehicle 4 can be achieved based on the comparison results via the management platform 31 to thereby enhance occupational safety and emergency response capability. Moreover, the actual images stored in the storage unit 323 and the comparison results stored in the management platform 31 can help clarify the facts when a traffic accident happens.

Therefore, for the vehicle driver, the control device 32 can compare the actual images with the predetermined conditions 3211 to obtain the comparison result and remind the vehicle driver when an abnormal state of the comparison result is caused by the reminding unit 324 timely to thereby reduce the incidence of traffic accidents caused when the vehicle driver does not pay attention to the surrounding environment. Further, the management platform 31 can provide the comparison result of each vehicle 4 timely to attain proactive monitoring of the driving state of each vehicle 4. Administrator can contact with the vehicle driver immediately when an abnormal state of the comparison result is shown to thereby handle a contingency properly and reduce the incidence of traffic accidents. Moreover, the performance of the vehicle driver can be evaluated through the comparison results to thereby achieve standardized management.

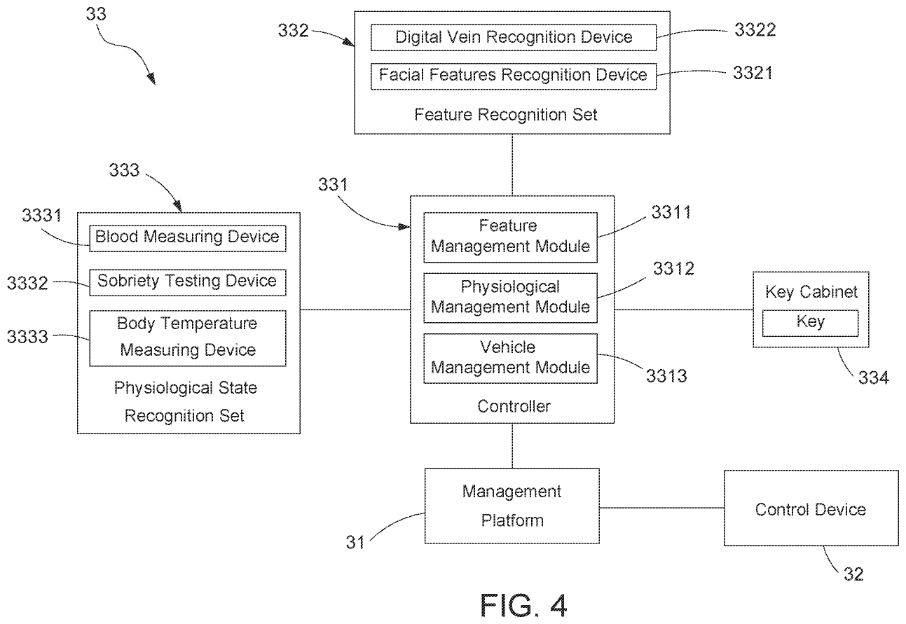

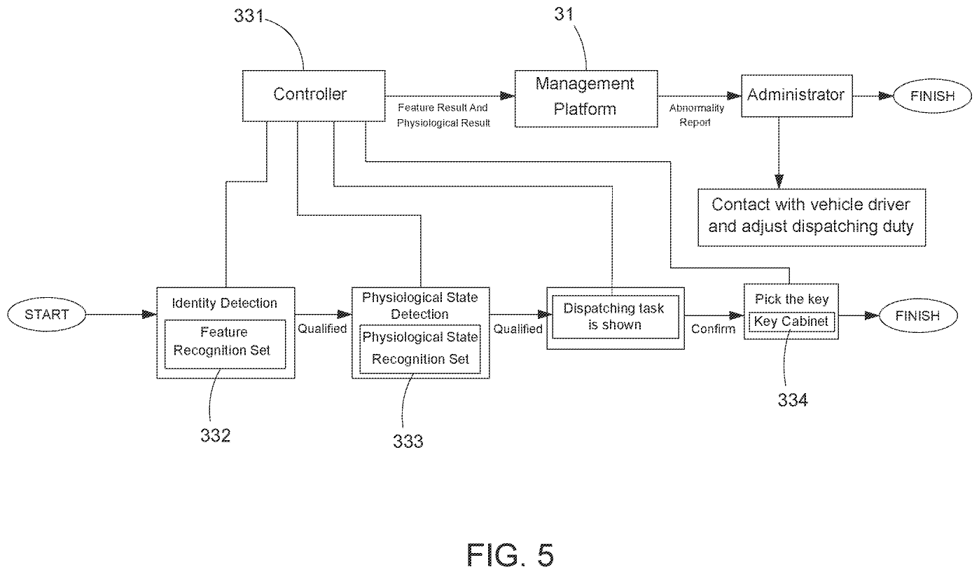

Referring to FIG. 4 and FIG. 5 show a second preferred embodiment of the vehicle monitoring system 3 of this invention. The correlated elements and the concatenation of elements, the operation and objectives of the second preferred embodiment are the same as those of the first preferred embodiment. This embodiment is characterized in that a vehicle dispatching device 33 is in communication with the management platform 31. The vehicle dispatching device 33 has a controller 331 in communication with the management platform 31, a feature recognition set 332 connected to the controller 331 and adapted to detect feature data of a vehicle driver, a physiological state recognition set 333 connected to the controller 331 and adapted to detect physiological data of the vehicle driver, and a key cabinet 334 controlled by the controller 331 and configured to accommodate at least one key. The controller 331 has a feature management module 3311 in which a plurality of feature default values are stored and a physiological management module 3312 in which a plurality of physiological default values are stored. The feature management module 3311 is connected to the feature recognition set 332 and adapted to receive the feature data detected by the feature recognition set 332. The feature default values are features of each vehicle driver that may include features of fingerprints and digital veins, facial features and so on. The physiological management module 3312 is connected to the physiological state recognition set 333 and adapted to receive the physiological data detected by the physiological state recognition set 333. The physiological default values are physiological values of each vehicle driver that may include values of blood alcohol content, blood pressure, blood oxygen, body temperature and so on.

The feature recognition set 332 can detect feature data of a vehicle driver. Here takes an example that the feature recognition set 332 has a facial features recognition device 3321 and a digital vein recognition device 3322 to detect the facial features and the feature of digital veins of the vehicle driver. After the feature recognition set 332 detects the features of the vehicle driver, feature data is obtained and transmitted to the feature management module 3311 of the controller 331. The feature management module 3311 then compares the feature data with the feature default values to obtain a feature result. When the feature data fulfills the feature default values, a normal state of the feature result is shown to thereby confirm an identity of the vehicle driver.

The physiological state recognition set 333 can detect physiological data of the vehicle driver. In this preferred embodiment, the physiological state recognition set 333 has a blood measuring device 3331, a sobriety testing device 3332, and a body temperature measuring device 3333 to thereby measure the blood pressure, blood oxygen, blood alcohol content and body temperature of the vehicle driver and obtain physiological data. The physiological data is then transmitted to the physiological management module 3312 of the controller 331. The physiological management module 3312 compares the physiological data with the physiological default values to obtain a physiological result. When the physiological data fulfills the physiological default values, a normal state of the physiological result is shown to thereby confirm a physiological state of the vehicle driver and determine that the physiological state of the vehicle driver is qualified for driving. The feature result and the physiological result are transmitted to the management platform 31 by the controller 331 and recorded in the management platform 31.

The management platform 31 can be set to report the feature result and the physiological result to the administrator. The administrator can also control the controller 331 via the management platform 31. When the feature data does not fulfill the feature default values, an abnormal state of the feature result is shown, and that may refer to a breakdown of the feature management module 3311, or masquerade of the vehicle driver. When the physiology data does not fulfill the physiological default values to show an abnormal state of the physiological result, that may refer to a poor physiological state of the vehicle driver such as high blood pressure, high blood oxygen, or high blood alcohol content that is not qualified for driving the vehicle 4. The management platform 31 monitors the physiological state and the identity of the vehicle driver to thereby keep tracking to the actual situation of the vehicle driver and execute after-treatment timely.

In this preferred embodiment, the controller 331 includes a vehicle management module 3313 in communication with the management platform 31. The vehicle management module 3313 is adapted to display a dispatching task to the vehicle driver after the feature data fulfills the feature default values to obtain a normal feature result and then the physiological data fulfills the physiological default values to obtain a normal physiological result. Thus, the vehicle driver can check his duty, the specific route for driving, and the specific vehicle 4 which he should pick according to the dispatching task. Finally, the key cabinet 334 is an electronic-controlled key cabinet. An opening operation and a close operation of the key cabinet 334 are controlled by the controller 331 according to the feature result and then the physiological result to allow the vehicle driver to pick the key of the specific vehicle 4 placed in the key cabinet 334.

Referring to FIG. 4 and FIG. 5, before the vehicle driver takes the key from the key cabinet 334, the feature recognition set 332 will detect the features of the vehicle driver to obtain feature data which is delivered to the feature management module 3311. The feature management module 3311 then compares the feature data with the feature default values to obtain a feature result. When the feature data fulfills the feature default values, an identity of the vehicle driver is confirmed. After that, the physiological state recognition set 333 detects the physiological state of the vehicle driver to obtain physiological data of the vehicle driver which is delivered to the physiological management module 3312. The physiological management module 3312 then compares the physiological data with the physiological default values to obtain a physiological result. When the physiological data fulfills the physiological default values, a physiological state of the vehicle driver is qualified for driving the vehicle 4. The feature result and the physiological result are transmitted and recorded in the management platform 31. The controller 331 then controls the key cabinet 334 to be open according to the normal feature result and then the normal physiological result to allow the vehicle driver to pick up the key of the vehicle 4. Thus, the vehicle dispatching process is completed. Therefore, when the vehicle driver attempts to pick up the key of a vehicle 4, the vehicle dispatching device 33 will detect an identity and a physiological state of the vehicle driver to thereby increase the driving safety. Further, the identity and the physiological state of the vehicle driver can be confirmed without any additional labor force to thereby reduce the labor costs. The management platform 31 is adapted to monitor the feature result and the physiological result to thereby proactively manage the actual situation of the vehicle driver directly and adjust the dispatching duty according to needs.

To sum up, the vehicle monitoring system of this invention takes advantages that the processor compares the actual images captured by the camera lens set with the predetermined conditions to obtain a comparison result which can show the actual driving state of each vehicle, and the reminding unit reminds the driver when the actual images does not fulfill the predetermined conditions to cause an abnormal state of the comparison result to thereby reduce the incidence of traffic accidents. The management platform monitors the driving state of each vehicle proactively according to the comparison result to thereby enhance occupational safety and emergency response capability. Moreover, the driving performance of the vehicle driver can be evaluated through the comparison results. Further, the vehicle dispatching device can be adapted to be in communication with the management platform to monitor an identity and a physiological state of the vehicle driver to thereby increase the driving safety and reduce the labor costs.

While the embodiments of this invention are shown and described, it is understood that further variations and modifications may be made without departing from the scope of this invention.

1. A vehicle monitoring system comprising:
a management platform adapted to monitor and manage a plurality of vehicles; and
a control device connected to said management platform and installed in each of said plurality of vehicles, said control device including:
a processor disposed in each of said plurality of vehicles and being in communication with said management platform,
a camera lens set disposed in each of said plurality of vehicles and connected to said processor,
a storage unit connected to said processor and adapted to store a plurality of actual images captured by said camera lens set, and
a reminding unit connected to said processor,
wherein a plurality of predetermined conditions are stored in said processor, said plurality of predetermined conditions being representative of standards for safe driving, said processor being configured to compare said plurality of actual images captured by said camera lens set with said plurality of predetermined conditions to obtain a comparison result serving to check a driving state of said vehicle, said reminding unit actuating an alarm responsive to said plurality of actual images failing to fulfill said predetermined conditions, said comparison result being delivered to said management platform by said processor and recorded in said management platform.

2. The vehicle monitoring system according to claim 1, further comprising an electronic control system installed in each of said plurality of vehicles and a signal line connected to said processor and said electronic control system for accessing vehicle driving data of said electronic control system and delivering said vehicle driving data to said processor.

3. The vehicle monitoring system according to claim 1, wherein said camera lens set includes a front camera, a left camera, a right camera, a rear camera, and an interior camera connected to said processor respectively for reflecting different sides of each vehicle, said plurality of actual images including a first image photographically captured by said front camera for reflecting a front side of said vehicle, a second image photographically captured by said left camera for reflecting a left side of said vehicle, a third image photographically captured by said right camera for reflecting a right side of said vehicle, a fourth image photographically captured by said rear camera for reflecting a rear side of said vehicle, and a fifth image photographically captured by said interior camera for reflecting a vehicle driver inside said vehicle.

4. The vehicle monitoring system according to claim 1, further comprising a vehicle dispatching device in communication with said management platform, said vehicle dispatching device including a controller in communication with said management platform, a feature recognition set connected to said controller and adapted to detect feature data of a vehicle driver, a physiological state recognition set connected to said controller and adapted to detect physiological data of said vehicle driver, and a key cabinet controlled by said controller and configured to accommodate at least one key, said controller including a feature management module in which a plurality of feature default values are stored and a physiological management module in which a plurality of physiological default values are stored, said feature management module being connected to said feature recognition set and adapted to receive said feature data detected by said feature recognition set and compare said feature data with said plurality of feature default values to obtain a feature result serving to check an identity of said vehicle driver, said physiological management module being connected to said physiological state recognition set and adapted to receive said physiological data detected by said physiological state recognition set and compare said physiological data with said plurality of physiological default values to obtain a physiological result serving to check a physiological state of said vehicle driver, an opening operation and a close operation of said key cabinet being controlled by said controller according to said feature result and then said physiological result, said feature result and said physiological result being sent from said controller to said management platform and recorded in said management platform.

5. The vehicle monitoring system according to claim 4, wherein said feature recognition set includes a facial features recognition device.

6. The vehicle monitoring system according to claim 4, wherein said feature recognition set includes a digital vein recognition device.

7. The vehicle monitoring system according to claim 4, wherein said physiological state recognition set includes a blood measuring device adapted to measure blood pressure and blood oxygen of said vehicle driver.

8. The vehicle monitoring system according to claim 4, wherein said physiological state recognition set includes a sobriety testing device.

9. The vehicle monitoring system according to claim 4, wherein said physiological state examination set includes a body temperature measuring device.

10. The vehicle monitoring system according to claim 4, wherein said controller includes a vehicle management module in communication with said management platform and adapted to display a dispatching task to said vehicle driver.
